(19) United States
(12) Patent Application Publication
(10) Pub. No.: US 2024/0099671 A1
(43) Pub. Date: Mar. 28, 2024

(54) CHASSIS FOR THE GANTRY OF A COMPUTED TOMOGRAPHY UNIT, GANTRY, AND COMPUTED TOMOGRAPHY UNIT

(71) Applicant: Siemens Healthcare GmbH, Erlangen (DE)

(72) Inventor: Hans-Juergen MUELLER, Pretzfeld (DE)

(73) Assignee: Siemens Healthcare GmbH, Erlangen (DE)

(21) Appl. No.: 18/472,533

(22) Filed: Sep. 22, 2023

(30) Foreign Application Priority Data
Sep. 23, 2022 (EP) 22197506.3

(51) Int. Cl. A61B 6/03; B33Y 80/00

(52) U.S. Cl. CPC A61B 6/035; B33Y 80/00

(57) ABSTRACT

One or more example embodiments of the present invention relates to a chassis for a gantry of a computed tomography unit, which has receiving areas to which rotating components of the computed tomography unit can be attached. The chassis is at least in part produced using additive manufacturing. One or more example embodiments of the present invention is also directed to a gantry and a computed tomography unit with such a chassis.

### CROSS-REFERENCE TO RELATED APPLICATION(S)

The present application claims priority under 35 U.S.C. § 119 to European Patent Application No. 22197506.3, filed Sep. 23, 2022, the entire contents of which are incorporated herein by reference.

### FIELD

One or more example embodiments of the present invention relates to a chassis for the gantry of a computed tomography unit, a gantry for a computed tomography unit, and a computed tomography unit.

### RELATED ART

In computed tomography (CT), sectional images of the patient are reconstructed from X-ray projections from a plurality of projection directions. To acquire the X-ray projections from different projection directions, an X-ray source and an X-ray detector situated opposite thereto rotate around a receiving area. In spiral CT, the patient to be examined is simultaneously guided through the tunnel of a gantry on a patient table. All rotating components, in particular the X-ray source and the X-ray detector, are attached to a chassis during the assembly of a computed tomography unit, said chassis being rotatably mounted in the gantry of the computed tomography unit. A chassis such as this is also referred to as a rotary frame, a drum or a rotatable part of the gantry. The gantry further typically has a fixed support frame, to which casing elements are attached. The chassis is rotatably mounted in this support frame, for example via a roller bearing or magnetic bearing.

To reduce the exposure to radiation for the patient as much as possible, and to prevent movement artifacts, efforts are made to enable a maximally high rotational speed of X-ray source and X-ray detector, for example up to 240 rpm. The chassis therefore has to satisfy the highest requirements as regards rigidity, so that the chassis does not deform even at high rotational speeds. Even slight deformations of just a few tenths of a millimeter can result in serious image artifacts if the relative position from the X-ray source to the X-ray detector does not remain exactly constant. In the prior art the chassis is hence designed as a cast aluminum or steel part with a relatively high wall thickness of approx. 20-25 mm. A cast part such as this supplies the necessary rigidity and a high resistive torque against load. In addition, in the prior art the chassis is typically pot-shaped or drum-shaped, so that the rotating components are kept stable even at high rotational speeds. A chassis such as this is disclosed for example in DE 10 2013 227 060 A1.

During each examination, the chassis together with the X-ray source and further rotating components is accelerated as fast as possible to the rotation time required for the examination, which correspondingly requires energy on the part of the drive motor of the chassis.

### SUMMARY

It is hence desirable to keep the mass to be rotated in a computed tomography unit as small as possible.

One or more example embodiments of the present invention provides a chassis for a gantry of a computed tomography unit (also called a CT device), which satisfies maximum requirements for rigidity at the same time as having the smallest possible weight. One or more example embodiments of the present invention further has the object of reducing the mass to be rotated in a computed tomography unit. One or more example embodiments of the present invention additionally has the object of providing a chassis with better attachment facilities for the rotating components of the computed tomography unit.

### BRIEF DESCRIPTION OF THE DRAWINGS

The invention is now described in greater detail using exemplary embodiments with reference to the accompanying drawings, in which:

FIG. 1 shows a schematic perspective representation of a computed tomography unit in accordance with an exemplary embodiment of the invention;

FIG. 2 shows a schematic plan view of a chassis in accordance with an exemplary embodiment of the invention;

FIG. 3 shows a perspective view of a chassis in accordance with the prior art;

FIG. 4 shows a perspective view of a chassis in accordance with a form of embodiment of the invention from a first side;

FIG. 5 shows a perspective view of a chassis in accordance with FIG. 4 from a second side;

FIG. 6 shows a schematic sectional view of a further form of embodiment of a chassis;

FIG. 7 shows a schematic sectional view of yet another form of embodiment of a chassis.

The same or similar elements are provided with the same reference characters in the figures.

### DETAILED DESCRIPTION

An inventive chassis has receiving areas, to which rotating components of the computed tomography unit can be attached and which is characterized in that it is produced at least in part using additive manufacturing. The term additive manufacturing, also called 3D printing, is in particular understood to mean manufacturing methods in which material is applied layer by layer in order to produce three-dimensional workpieces. This typically happens on a computer-controlled basis from materials in liquid or powder form in accordance with a CAD plan, so that any desired three-dimensional (3D) shapes can be achieved.

Thanks to at least partial production via additive manufacturing it is possible to optimize the structure of the chassis in the direction of lightweight construction, at the same time as ensuring maximum inherent stability. In accordance with one or more example embodiments of the present invention it is therefore possible for example to provide a 3D-printed chassis with a structure-optimized design. Thanks to the high degree of flexibility in the design of the chassis it is possible to achieve a low weight, in particular thanks to small wall thicknesses and/or the use of material with a low specific weight, and nevertheless still to provide sufficient rigidity or inherent stability for the requisite high speeds, or even for still higher speeds than are currently the norm. In particular, the inventive chassis is suitable for rotational speeds of over 260 rpm, preferably approximately 280-320 rpm.

The reduction in weight of the chassis further has the advantage that a lower static load acts on the rotary bearing of the gantry. Furthermore, the total weight of the gantry and thus also the transportation costs and the floor loading at the customer's premises are reduced. Above all, less rotational and acceleration energy is required, since the weight of the rotating unit (chassis and rotating components) is reduced. As a result, a smaller rotation motor is possible and/or a faster acceleration to the rated rotational speed, which in turn is reflected in a shorter waiting time for patients and a higher patient throughput. Because of the low weight a computed tomography unit with the inventive chassis has higher sustainability. In addition, thanks to the low weight the friction at the bearings is also reduced, which in turn increases the service life of the computed tomography unit.

When the chassis is produced using additive manufacturing the topology or structure of the chassis can be optimized using an algorithm. As a result, even unconventional, for example non-rotationally symmetrical, topology can be calculated, which at a specified high rotational speed withstands expected stresses. Safety elements can also be specified in the calculation, for example so that a complete failure does not occur if there is a local crack in the chassis. One or more example embodiments of the present invention therefore provides a chassis with a structure-optimized geometry which offers sufficiently rigid structures even for higher rotational speeds.

The term “rotating components” of the computed tomography unit is understood to mean all those components that rotate around the patient during the acquisition of the CT data, in particular the X-ray source and the X-ray detector. In the assembled CT device the rotating components are typically attached to the chassis. Further rotating components can for example be filters and collimators attached in front of the X-ray tube, cooling for the X-ray tube unit, a voltage generator, and further electronic devices needed to operate the X-ray tube and X-ray detector, in so far as these cannot be arranged to be stationary outside the chassis, but have to be connected directly to the X-ray source and X-ray detector so that these can be operated. The term receiving area for a rotating component is understood to mean the area in which this component is to be arranged in the assembled CT device and attached to the chassis.

The chassis can be produced at least in part via one of the following additive manufacturing methods: Fused Deposition Modeling (FDM), Selective Laser Melting (SLM), Selective Laser Sintering (SLS), Electron Beam Melting (EBM), Directed Energy Deposition (DED).

A preferred method is the DED method, since it delivers maximum flexibility of design and at the same time is suitable for many materials, also including metals. Moreover, it is also suitable for hybrid applications, thus for example for positioning elements on workpieces that are already present. In DED use can be made of a high-powered laser in order to melt metal powder, which is supplied to the focus of the laser beam. The laser beam generally runs through the center of a coating head and is focused by one or more lenses onto a small point. The metal powder is supplied and distributed over the circumference of the coating head or can be supplied by nozzles that are arranged around the coating head. The buildup takes place on a table that can move in a plane and is moved on a track created on the basis of a digital model, in order to produce an object layer by layer. The coating head is moved vertically upward when each layer is complete. Some systems even use 5-axis or 6-axis systems (i.e. articulated arms), which are able to apply material to the workpiece with few if any spatial access restrictions. The DED method is suitable for a broad range of materials such as titanium, stainless steel, aluminum, tungsten and other special materials as well as for composite materials. Using the method, not only can new workpieces be produced in full, but material can also be added to existing parts, for example for hybrid manufacturing applications.

Selective laser sintering (SLS) is also very suitable. In this case a laser is used as a heat source in order to sinter material in powder form (e.g. nylon or polyamide), the laser being automatically directed at points in space that are defined by a 3D model, the laser joining the material to form a permanent structure. For this there are rapid 3D printing methods, in which the workpiece is however still sintered after printing in order to achieve a sufficiently permanent and rigid connection.

Additive manufacturing methods for plastics are further possible, in which the chassis is built up at least in part from fiber-reinforced plastic.

The chassis can be manufactured at least in part from one of the following selection of materials: steel, a steel alloy, aluminum, an aluminum alloy, fiber-reinforced plastic, a titanium alloy, a chromium-cobalt alloy or a magnesium alloy. It is preferably manufactured at least in part from a material that is suitable for additive manufacturing and/or is relatively light, such as for example aluminum and aluminum alloys and/or fiber-reinforced plastic (FRP), in particular glass-fiber-reinforced plastic (GFRP) or carbon-fiber-reinforced plastic (CFRP). GFRP or CFRP can be processed via additive manufacturing such that the fibers run in the direction of force, so that they are particularly suitable for the production of reinforcement elements, struts or bracing elements using lightweight construction.

In accordance with one form of embodiment the chassis has struts produced via additive manufacturing. A strut is for example a stanchion, bar or bracing element that connects two points of the chassis together in a force-fit manner. In particular, a strut can absorb forces along its longitudinal direction. One or more recesses can be situated between the strut and other areas of the chassis. By using struts a particularly high inherent stability can be achieved with a maximally low weight, but they are not easy to produce using the conventional production methods for the chassis, such as aluminum sand casting. This is the case above all if the struts are to be provided in multiple planes. In contrast, via additive manufacturing struts can be manufactured in any desired shapes and directions.

In accordance with a preferred form of embodiment the chassis has at least one strut produced via additive manufacturing which connects two points to one another on an annular element arranged around the axis, in particular on the outer circumference of the chassis, in particular in the manner of a tendon, wherein the strut need not be straight. The strut can for example curve outward, viewed from the axis. Thanks to a strut such as this a high inherent stability of the chassis, above all on its outer circumference, can be achieved even at high rotational speeds. Two to three such struts are advantageously provided, which for example in each case connect points to one another at an angular spacing of between 90 and 170 degrees.

The chassis is typically rotatable about an axis, which in the assembled CT device usually runs horizontally through the center point of the gantry. The rotating components of the CT device can be arranged approximately annularly around this axis. For this purpose, conventional chassis produced from aluminum sand casting often have a baseplate which has the basic shape of a round perforated disk and at the outer circumference transitions into an approximately cylindrical drum which encloses the rotating components in the circumferential direction. However, a cast part cannot, starting from the baseplate, have any undercuts, since otherwise it would be necessary to work with a lost mold. The drum cannot therefore curve inward again in order for example to hold one of the rotating components from two sides.

In contrast, this is possible with the invention, for which reason the inventive chassis has at least one undercut in accordance with one form of embodiment. This can mean that sections of the chassis thicken and/or curve in the axial direction, but in any case do not remain within their footprint in the axial direction. An undercut can also be a section of the chassis that encloses a receiving area for a rotating component of the computed tomography unit at least in part, so that this is in particular enclosed from two opposing sides.

An undercut can contribute to a receiving area such as this being encompassed at least in part from two sides by sections of the chassis, which enables a particularly stable attachment of the rotating component. In particular, it can be provided that a receiving area can be surrounded in the axial direction and/or in the circumferential direction and/or in the radial direction from two sides by sections of the chassis, particularly preferably in the axial direction and/or in the radial direction.

The chassis can have the shape of a hollow body, in which in particular receiving areas can be surrounded by walls and/or struts of the chassis. In particular, the chassis can have approximately the shape of an inwardly open torus, wherein the outer surface of the torus is preferably provided with recesses and/or is formed at least in part by struts or bracing. From the inside, in other words from the axis outward, the rotating components can be inserted into the chassis. The torus need not be spanned by a circle, but in cross-section can also have an approximately oval, square, parallelogram-shaped or rectangular shape, where appropriate with rounded corners. A body such as this, essentially formed from struts or bracing, is also referred to as a “bird's nest”.

The chassis can also have the shape of a hollow ring or tire, wherein this shape too preferably has walls and/or struts or bracing provided with recesses.

In accordance with one form of embodiment the chassis is lightweight in construction, in particular with an average wall thickness of up to 30 mm, preferably 5 mm to 20 mm, even more preferably 10-18 mm. Thanks to additive manufacturing it is possible to optimize the structure of the chassis such that its walls and struts can be designed with a very small wall thickness, without forgoing rigidity. On the one hand the struts and on the other hand the optimized basic shape, such as for example the tire or bird's nest shape, can contribute to this.

In accordance with one form of embodiment the chassis weighs 20%-60%, preferably 30%-50%, less than a corresponding chassis made of cast aluminum in accordance with the prior art.

In accordance with one form of embodiment the chassis weighs less than 200 kg, preferably less than 160 kg, even more preferably 100-140 kg. These specifications refer to a chassis with an internal diameter of 60-100 cm, preferably 70-90 cm, in particular 80 mm, and an outer diameter of 1500 mm to 18000 mm, in particular 1600 mm to 1700 mm, e.g. 1650 mm, and without the rotating components. A chassis of this size produced conventionally using aluminum sand casting in contrast has a mass of approx. 240 kg. In the case of smaller chassis, for example for head computed tomography units, there is a similarly large percentage weight saving potential. The weight saving achievable using additive manufacturing and the optimized structure is therefore considerable.

In accordance with one form of embodiment the chassis has a frame not produced by additive manufacturing and further elements produced by additive manufacturing. As a result, the advantages of conventional and additive production methods can advantageously be combined with one another. For example, it is possible to produce a frame inexpensively, e.g. from a rolled metal element, in particular a rolled flat plate made of aluminum alloy or steel. The frame can for example have the basic shape of a round perforated disk which is stamped from a rolled metal product. Alternatively the frame can also be a cast part made of metal or plastic, or can be manufactured from a continuously cast metal element. Finally the frame can also be manufactured from a cylindrical round-rolled or continuously cast metal part. The fame can additionally be mechanically machined, e.g. by turning or milling, for example to obtain recesses.

In accordance with one form of embodiment of the invention, further elements produced using additive manufacturing are attached to a “basic frame” such as this, and in particular contribute to the stabilization and inherent stability of the chassis. This can be done in that the further elements are produced separately using additive manufacturing and are then connected to the frame, for example by welding, gluing, by form-fit, by connection elements such as screws or rivets, or a combination of these connections. This has the advantage that the further elements can be produced particularly inexpensively and that it provides particularly large amounts of degrees of freedom in terms of design. The connected elements are in particular struts. It is also possible to position the further elements directly onto the frame using additive manufacturing. This has the advantage that a further connection step is saved. In this case the frame can be clamped into the tool for the additive manufacturing and the further elements can be built up directly onto the base frame, for example using DED.

In accordance with an alternative form of embodiment the chassis is completely produced using additive manufacturing. This permits a very extensive optimization of the construction. The chassis can be produced in one piece, but it can also be constructed from multiple elements connected to one another, wherein the individual elements are in turn produced using additive manufacturing. This permits the combination of different materials, in particular of metal with FRP, which permits a particularly weight-saving construction.

In accordance with one form of embodiment the chassis comprises multiple elements connected to one another, of which at least one is produced using additive manufacturing. For example, one or more elements produced using additive manufacturing, such as struts, can be connected to a frame not produced using additive manufacturing, as described above. It is advantageous for at least 20%, preferably 35%, even more preferably 50% of the mass of the chassis to be produced using additive manufacturing.

In accordance with one form of embodiment the chassis has bracing elements which enclose the at least one receiving area for a rotating component of the CT device on at least one side, preferably on two or three sides. The bracing can in particular be a bar arrangement, the bars of which are connected to one another at their ends at node points. The bars are advantageously predominantly or completely stressed by forces along their longitudinal direction, in particular are subject to tension. As a result, by using a small amount of material a particularly high inherent stability of the chassis can be achieved. The bracing elements or a bracing formed by these can form a wall of the chassis, in particular a wall on an outer circumferential surface of the chassis, or a wall in the form of a flat or curved perforated disk, which approximately assumes the function of the baseplate of the conventional chassis.

In accordance with one form of embodiment the chassis has at least one continuous ring, on which elements produced using additive manufacturing, in particular struts, are positioned. The ring can likewise be produced using additive manufacturing, or can also be manufactured conventionally for example as a cast part or as a continuously cast and/or bent part. The ring advantageously defines the internal diameter of the chassis. Its axis can correspond to the axis of the chassis. The bearing on which the chassis is rotatably mounted can be located on such an inner ring in the assembled CT device. Struts can in particular run radially outward from such an inner ring, where appropriate with a curvature toward the axial direction, in order to form a receiving area for a rotating component. In accordance with an advantageous form of embodiment at least some of these struts are again connected to a ring at their other end, which in particular runs coaxially to the inner ring. This ring can be arranged on the outer circumference of the chassis, in other words in the radial direction outward. This outer ring can externally enclose the rotating components, and where appropriate rotating components can also be attached to the outer ring. The outer ring can be arranged offset in the axial direction compared to the inner ring. In accordance with one form of embodiment, struts are positioned on the outer ring, and stabilize the outer ring, in particular prevent its deformation at high rotational speeds. These can be tendon-like struts, which in each case connect together two points on the outer ring spaced apart in the circumferential direction by 90° to 170°. Such tendon-like struts can in turn be connected to further struts that run to the outer ring. The tendon-like struts can enclose a receiving area on a side opposite the inner ring, thus enabling rotating components to be held securely.

The inner and/or outer ring of the chassis can be produced via additive manufacturing. However, it can also be produced using a conventional method, for example bending of metal bars or stamping from a rolled flat metal product. The above-mentioned struts are preferably produced via additive manufacturing, in particular positioned onto the inner and/or outer ring.

In accordance with one form of embodiment the chassis has a rigidity such that during a rotation in operation of the computed tomography unit it is maximally deformable by up to 0.3 mm, preferably up to 0.2 mm. The term rotation here means a rotational speed of at least 240 rpm, preferably at least 300 rpm. In accordance with the prior art, at such rotational speeds a considerably greater deformation is to be expected, in particular up to 0.6 mm or even more.

One or more example embodiments of the present invention is also directed to a gantry for a computed tomography unit with a chassis as described herein, to which are attached rotating components of the computed tomography unit, in particular an X-ray source and an X-ray detector unit. The gantry typically has normal dimensions, and in addition to the chassis and the rotating components inserted therein can also have a support frame and a casing.

In accordance with a further aspect one or more example embodiments of the present invention is also directed to a computed tomography unit which has an inventive chassis and/or an inventive gantry. The computed tomography unit can further have a gantry as described above. Further components such as for example a computer for the reconstruction of the acquired data and an operator console can likewise be part of the computed tomography unit. All advantages and exemplary embodiments described with reference to the chassis also apply to the gantry and the computed tomography unit, and vice versa.

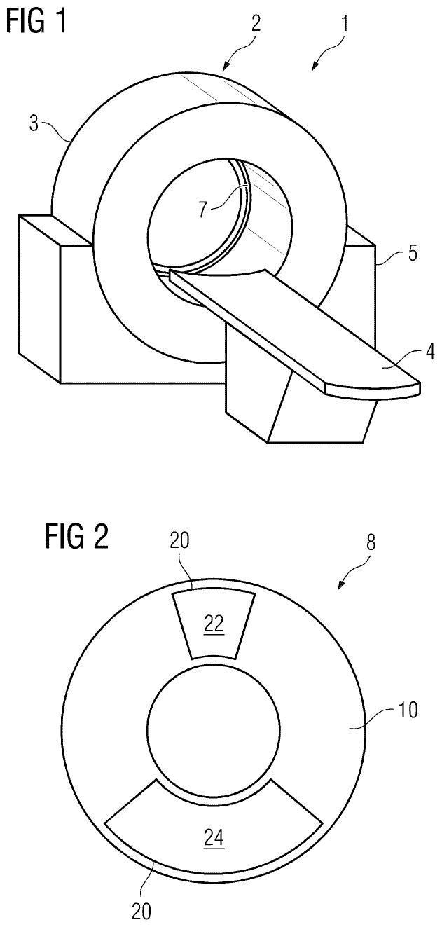

FIG. 1 shows in simplified form a CT device 1 with a gantry 2, in which an inventive chassis 30 is housed. This is mounted on a support frame 5. The gantry 2 is provided with a casing 3. It has an annular opening, through which the patient table 4 can be pushed. Around the annular opening runs a ring 7, on which the chassis is mounted, for example by a roller bearing, so that it can rotate about a patient positioned on the patient table 4. The drive mechanism for the rotation is not shown.

FIG. 2 shows a schematic plan view of a chassis 8 in accordance with the prior art. The chassis has a baseplate 10, to which the rotating components 20 are attached. The rotating components 20 of the computed tomography unit are in particular an X-ray tube 22 and an X-ray detection unit 24. Further components, which are not shown in FIG. 2 but which can be arranged around the further circumference of the chassis 8, are for example high-voltage generators and cooling systems.

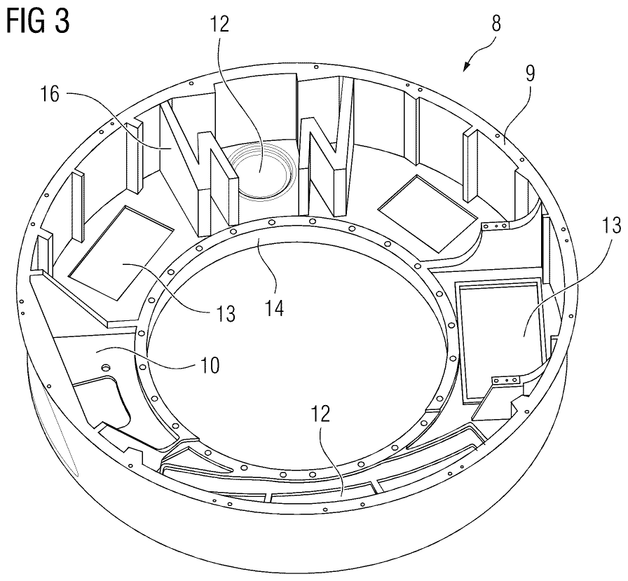

FIG. 3 shows a chassis 8 in accordance with the prior art. This is a metal casting, in particular it is produced by aluminum sand casting. The chassis 8 has a baseplate 10 which has the basic shape of a round perforated disk, but which is provided with recesses 13. A drum-like, continuous wall 9 is provided on the outer circumference. This wall 9 has a relatively large wall thickness of over 20 mm, since it must absorb high forces during the rotation of the chassis 8 with the rotating components 20, without being deformed too much, since otherwise the X-ray tube 22 and the detector unit 24 lose their position relative to one another. A lower projection 14 is provided on the inner radius of the chassis 8. In the radial direction two dividing walls 16 are arranged on the baseplate 10, and separate the individual receiving areas 12 for the rotating components of the gantry from one another. Thus the chassis 8 forms receiving areas 12 for rotating components 20 of the computed tomography unit, which in particular are surrounded by the walls 9, 14 and 16. Because of the production using sand casting it is however not possible for the outer wall 9 for example to curve inward again or to form an undercut, although this would be helpful for defining the rotating components 20. In addition the chassis 8 has a very high weight.

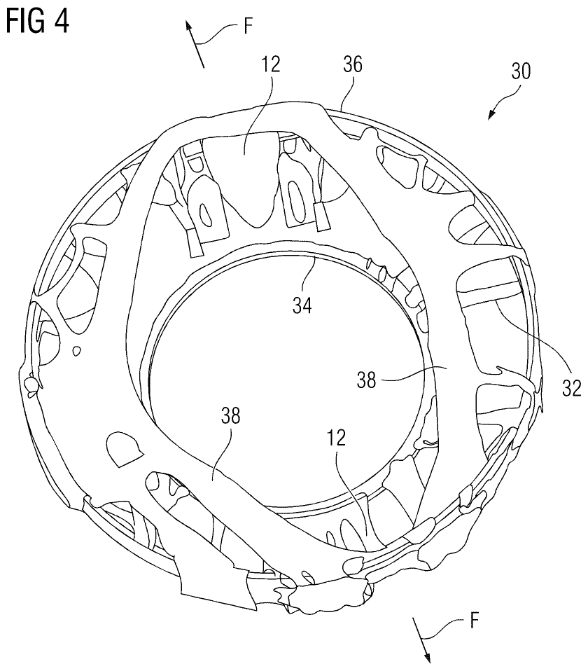

FIG. 4 shows a chassis 30 in accordance with an exemplary embodiment of the invention. This is effected using lightweight construction via additive manufacturing. It has an inner ring 34, which has a diameter such that the mounting on the ring 7 of the gantry can take place on it. An outer ring 36 is further provided, on which the bracing-like structure is attached to the outer circumference of the chassis 30. Struts 32 in irregular shapes and thicknesses run between the inner ring 34 and the outer ring 36. These shapes and thicknesses have been calculated in accordance with the weight of the rotating components 20 to be inserted, in order to ensure an optimum rigidity and stability with the least possible weight. In addition, two tendon-like struts 38 are arranged on the outer ring 36, which curve over the receiving areas for the rotating components and support the outer ring 36 and protect it against deformation. During a rotation of the chassis 30 with the inserted rotating components 20 and at a high speed, forces are particularly to be expected in the direction of the arrows F, which without the struts 38 would result in a deformation of the chassis 30, in particular of the ring 36 on the outer circumference. Thanks to the production via additive manufacturing it is now possible to provide reinforcement elements, such as struts for example, in two or more planes spaced apart from one another in the axial direction, as an example in FIG. 4 the struts 32 and 38. The resulting shape is also referred to as a bird's nest. The chassis 30 in FIG. 4 is completely produced via additive manufacturing. However, it is possible to produce the inner ring 34 and where appropriate also the outer ring 36 conventionally, and then to use hybrid technology to position the intermediate elements, in particular the struts 32 and 38, onto the rings via additive manufacturing.

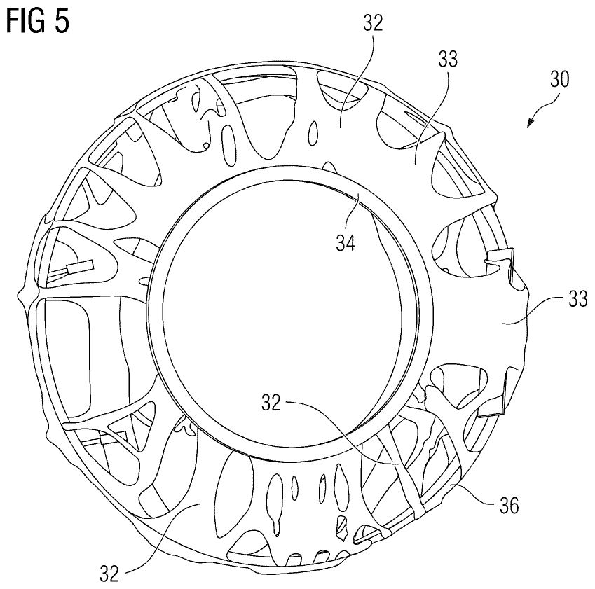

FIG. 5 shows the chassis 30 in FIG. 4 from the other side. The numerous struts 32, which are irregularly shaped and in this case form a type of bracing that connects the inner ring 34 to the outer ring 36, can be seen particularly well here. The struts 32 are in part also designed as flat, as can be seen for example in the case of 33.

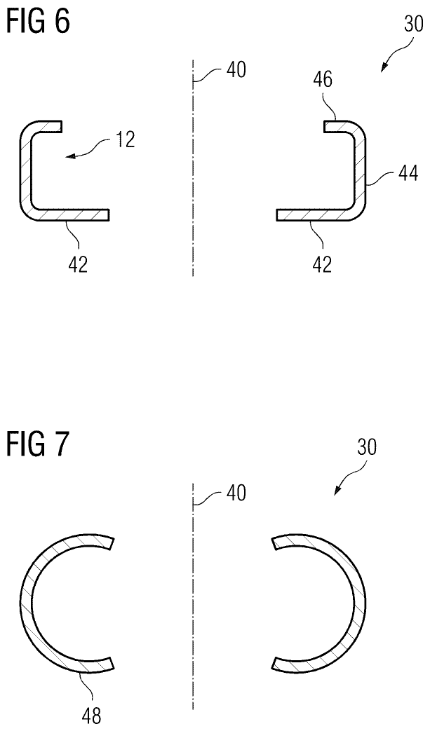

FIG. 6 shows a cross-section along the axis 40 through a chassis 30 in accordance with another form of embodiment. This chassis 42 has a base frame 42 which has approximately the shape of a round perforated disk. In the radial direction outward a drum-shaped wall 44 is then positioned which, offset in the axial direction from the base frame 42, curves inward again at 46. As a result, an undercut is formed at 46, and a rotating component 20 arranged in the receiving area 12 is held securely. In other words, a rotating component 20 can be encompassed from two sides by elements of the chassis, namely by the base frame 42 and the undercut 46. Both the base frame 42 and the drum-like part 44 and the section 46 are preferably essentially formed from struts or bracing.

FIG. 7 shows a cross-section along the axis 40 through a chassis in accordance with yet another form of embodiment. In this case the chassis 30 has the shape of an inwardly open torus or tire 48. Here too, the wall 48 of the chassis is preferably formed from struts, which in particular form a bracing.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

1. A chassis for a gantry of a computed tomography unit, the chassis comprising:
receiving areas, to which rotating components of the computed tomography unit can be attached, wherein the chassis is produced at least in part using additive manufacturing.

2. The chassis of claim 1, further comprising:
struts formed via additive manufacturing.

3. The chassis of claim 1, further comprising:
undercuts formed via additive manufacturing.

4. The chassis of claim 1, wherein the chassis includes an average wall thickness of up to 20 mm.

5. The chassis of claim 1, characterized in that the chassis weighs less than 200 kg.

6. The chassis of claim 1, further comprising:
a frame not produced using additive manufacturing; and
at least one other element produced using additive manufacturing.

7. The chassis of claim 6, wherein the frame is produced from a rolled metal element.

8. The chassis of claim 1, wherein the chassis is entirely produced using additive manufacturing.

9. The chassis of claim 1, further comprising:
bracing elements, the bracing elements enclosing at least one receiving area on at least one side.

10. The chassis of claim 1, further comprising:
at least one continuous ring; and
struts positioned on the at least one continuous ring via an additive manufacturing process.

11. The chassis of claim 1, wherein the chassis has a rigidity, such that during a rotation in operation of the computed tomography unit it is maximally deformable by up to 0.3 mm.

12. A gantry for a computed tomography unit with the chassis of claim 1.

13. A computed tomography unit, comprising:
the chassis of claim 1.

14. The chassis of claim 2, further comprising:
undercuts formed via additive manufacturing.

15. The chassis of claim 14, wherein the chassis includes an average wall thickness of up to 20 mm.

16. The chassis of claim 15, characterized in that the chassis weighs less than 200 kg.

17. The chassis of claim 16, further comprising:
a frame not produced using additive manufacturing; and
at least one other element produced using additive manufacturing.

18. The chassis of claim 17, wherein the frame is produced from a rolled metal element.

19. The chassis of claim 18, further comprising:
at least one continuous ring; and
struts positioned on the at least one continuous ring via an additive manufacturing process.

20. The chassis of claim 19, wherein the chassis has a rigidity, such that during a rotation in operation of the computed tomography unit it is maximally deformable by up to 0.3 mm.
